(19) United States
(12) Patent Application Publication
(10) Pub. No.: US 2023/0259866 A1
(43) Pub. Date: Aug. 17, 2023

(54) Diagnostic Kit and A Computer Implemented Method and System for Managing Order Fulfilment of The Diagnostic Kit

(71) Applicant: Thriva Limited, London (GB)

(72) Inventors: Thomas James Livesey, London (GB); Matthew David Krog, London (GB); Myles Philip Cowper-Coles, London (GB)

(21) Appl. No.: 17/677,616

(22) Filed: Feb. 22, 2022

(30) Foreign Application Priority Data
Feb. 15, 2022 (GB) 2201954.1

(51) Int. Cl. G06Q 10/08; G16H 10/40; G16H 40/67

(52) U.S. Cl. CPC G06Q 10/0832; G16H 10/40; G16H 40/67

(57) ABSTRACT

The present invention relates to a diagnostic kit and a computer implemented method and system for managing order fulfilment of said diagnostic kit. The method comprises the step of scanning, by a fulfilment entity, an identifier provided on a packaging of a diagnostic kit, the identifier being linked to stored personal data of a person to whom the diagnostic kit is to be delivered; wherein the scanning of the identifier provides the fulfilment entity access only to delivery details of the stored personal data of the person so that the diagnostic kit can be delivered to the person

### FIELD OF THE INVENTION

The invention relates to the field of order fulfilment management, and particularly but not exclusively, to the management of order fulfilment of a diagnostic kit.

### BACKGROUND OF THE INVENTION

Despite traditional diagnostic procedures which typically require biological specimen sampling from test subjects at specified locations such as medical clinics, health centers, hospitals or the like where the procedure will generally be carried out by medical professionals, there have been increasing demands for “self-test” diagnostic products which can be used by the test subject to conduct the sampling or testing at home or in a similar environment. This is particularly in view of the recent global pandemic where regular testing for diseases such as the coronavirus disease (Covid-19) at home is preferred to reassure that one is not infected with the disease before going to school or work, or for people who are under quarantine at home or at an isolation or quarantine facility. Common home-use diagnostic kits typically require users to register the kit with their personal information before sending the kit with the collected specimen to the testing facilities such as laboratories or clinics. In most circumstances, registration can be done either manually by entering by the user a serial number of the test kit via an online form, or by scanning a barcode provided on the test kit, and subsequently, registering the serial number or the barcode with the user's information. In the case where more than one test is being done or regular testing is required, the user may have to register each of the test kits under the same procedure, or to register the test kit each time before testing. After the self-sampling is completed, the test kit with the sampled specimen will be shipped, such as by postal or courier services, to the testing laboratory. The technician at the laboratory receiving the test kit will then associate the specimen with the user's identity based on the registered user information. The test results will then be sent to the user or to the user's medical practitioner or even to relevant governing bodies based on the user's personal information. In the scenarios where electronic registration is not available, the user's personal information may have to be attached, such as in paper form, along with the test kit over the order and/or testing fulfillment process. It therefore raises privacy concerns as to how the fulfilment service providers such as the courier service providers and/or the test service providers may handle sensitive personal data of the users.

### OBJECTS OF THE INVENTION

An object of the invention is to provide a novel solution for managing order and/or test fulfilment of a product such as, but is not limited to, a diagnostic kit or similar product.

Another object of the invention is to mitigate or obviate to some degree one or more problems associated with known solutions in the prior art.

The above object is met by the combination of features of the main claims; the sub-claims disclose further advantageous embodiments of the invention.

One skilled in the art will derive from the following description other objects of the invention. Therefore, the foregoing statements of object are not exhaustive and serve merely to illustrate some of the many objects of the present invention.

### SUMMARY OF THE INVENTION

In a first main aspect, the invention provides a computer implemented method for managing order fulfilment of a diagnostic kit. The method comprises scanning, by a fulfilment entity, an identifier provided on a packaging of a diagnostic kit, the identifier being linked to stored personal data of a person to whom the diagnostic kit is to be delivered; wherein the scanning of the identifier provides the fulfilment entity access only to delivery details of the stored personal data of the person so that the diagnostic kit can be delivered to the person.

In a second main aspect, the invention provides a system for managing order fulfilment of a diagnostic kit. The system comprises a scanning module for scanning an identifier provided on a packaging of a diagnostic kit, a memory for storing data, and a processor for executing computer readable instructions, wherein the processor is configured by the computer readable instructions when being executed to implement the method according to the first main aspect.

In a third main aspect, the invention provides a non-transitory computer readable medium storing machine-readable instructions which, when executed on a processor, implement the method according to the first main aspect.

In a fourth main aspect, the invention provides a diagnostic kit. The diagnostic kit comprises a packaging where the diagnostic kit is packed, the packaging being provided with an identifier linked to stored personal data of a person to whom the diagnostic kit is to be delivered; wherein scanning of the identifier provides a fulfilment entity access only to delivery details of the stored personal data of the person so that the diagnostic kit can be delivered to the person.

### BRIEF DESCRIPTION OF THE DRAWINGS

The foregoing and further features of the present invention will be apparent from the following description of preferred embodiments which are provided by way of example only in connection with the accompanying figures, of which:

FIG. 1 is a schematic diagram showing a packed diagnostic kit having an identifier on the packaging in accordance with an embodiment of the present invention;

FIG. 2 is a block diagram showing the system in accordance with an embodiment of the present invention; and

FIG. 3 is a flow diagram showing the method steps as implemented by an embodiment of the present invention.

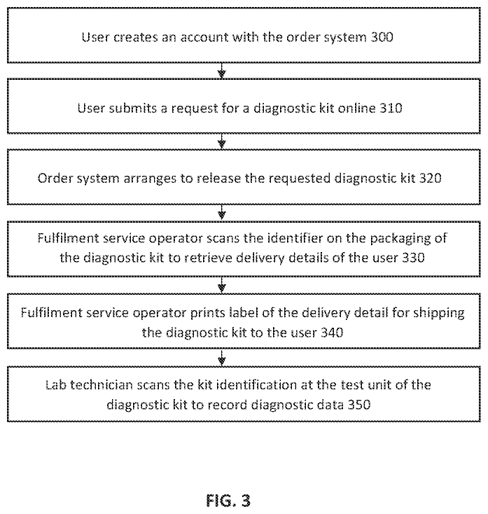

### DESCRIPTION OF PREFERRED EMBODIMENTS

The following description is of preferred embodiments by way of example only and without limitation to the combination of features necessary for carrying the invention into effect.

Reference in this specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments, but not other embodiments.

It should be understood that the elements shown in the figures may be implemented in various forms of hardware, software, or combinations thereof. These elements may be implemented in a combination of hardware and software on one or more appropriately programmed general-purpose devices, which may include a processor, memory, and input/output interfaces.

The present description illustrates the principles of the present invention. It will thus be appreciated that those skilled in the art will be able to devise various arrangements that, although not explicitly described or shown herein, embody the principles of the invention and are included within its spirit and scope.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Thus, for example, it will be appreciated by those skilled in the art that the block diagrams presented herein represent conceptual views of systems and devices embodying the principles of the invention.

The functions of the various elements shown in the figures may be provided using dedicated hardware as well as hardware capable of executing software in association with appropriate software. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (“DSP”) hardware, read-only memory (“ROM”) for storing software, random access memory (“RAM”), and non-volatile storage.

In the claims hereof, any element expressed as a means for performing a specified function is intended to encompass any way of performing that function including, for example, a) a combination of circuit elements that performs that function or b) software in any form, including, therefore, firmware, microcode, or the like, combined with appropriate circuitry for executing that software to perform the function. The invention as defined by such claims resides in the fact that the functionalities provided by the various recited means are combined and brought together in the manner which the claims call for. It is thus regarded that any means that can provide those functionalities are equivalent to those shown herein.

Reference herein to a “diagnostic kit” is to be taken as a reference to a “test kit” and vice-versa.

The invention generally relates to a diagnostic product such as a diagnostic kit, and a method and a system for managing order and/or testing fulfilment of said product. The diagnostic kit can be a so-called “self-test” or “home-test” kit for one to carry out testing or sampling by himself or herself at home. Yet the invention should not be restricted to application for diagnostic products only, but instead, it will be understood that the invention is applicable for managing order fulfilment of other product types not necessarily related or limited to medical and/or healthcare products if an order fulfilment process is required. In the context of the present invention, the term “fulfilment” may generally relate to one or more processes covering picking, labelling, linking, dispatching and/or returning, etc. of a product unit.

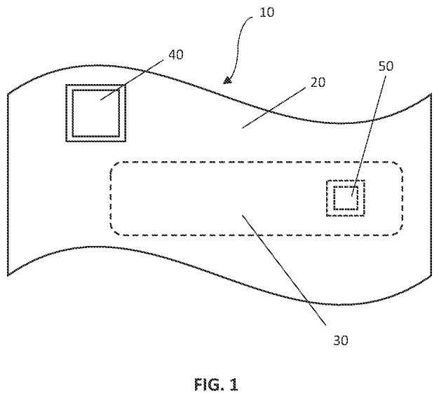

Referring to FIG. 1, shown is an exemplary embodiment of a packaged diagnostic kit 10 which is packed with or comprises a packaging 20. The packaging 20 can be any type of common protective packaging for containing a test unit 30. Particularly, the packaging 20 is provided with an identifier 40, which can be printed on, adhered to in the form of a printed label, or in any way connected or associated with the packaging 20 such as being provided in the form of a tag. The identifier 40 is preferably provided on or located at an exterior of the packaging 20, such as an external front side of the packaging, where it is easily observable or accessible by a person handling the packaged diagnostic kit 10 for delivery to a user, for example. In one embodiment, the identifier 40 may comprise a two-dimensional code, such as one or more of a quick response (QR) code, a bar code and/or any equivalent. Preferably, the identifier 40 is a unique identifier.

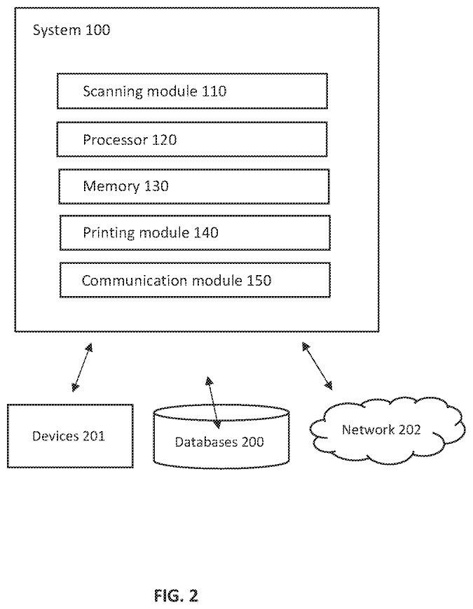

FIG. 2 shows by way of example is a block diagram of a computer implemented system 100 in accordance with concepts of the present invention. Without being limited by any specific embodiments described and illustrated, the system 100 can be provided in the form of any computer implemented software such as a computer program, platform, or application, etc. Alternatively, the system 100 can be implemented at, provided as, or configured to comprise a computer processing device comprising a processor 120 for executing computer readable instructions stored in a memory to implement the method steps of the present invention. In another embodiment, the elements as described and shown can be implemented in or as various forms of hardware, software, or a combination thereof. In the context of the present invention, the terms “computer”, “computer device”, “computer processing device”, “processor”, “processing device” are intended to encompass any suitable processing device. For example, any components, systems, or devices described herein can be associated with any computer or processing devices such as general-purposed computers, client terminals, or other suitable devices such as mobile phones, smart phones, tablets, smart watches, or any other mobile computing devices. In one embodiment, the system 100 may comprise, or be executed via the processor 120 which comprises, a plurality of functional blocks for performing various functions thereof. For example, the system 100 may comprise a scanning module 110 which can be provided as or connected with a scanner or similar reading device for scanning or reading the identifier 40 at the packaging 20 of the diagnostic kit 10; a memory 130 for storing data; and a communication module 150 for transmitting and receiving instructions, information and/or data to and from the system 100 such as, but not limited to, a database 200, one or more computer devices 201, and/or a communication network 202. In one embodiment, the communication network 202 may comprise a single network, but may also comprise multiple interconnected networks such as one or more internal and external networks. In one further embodiment, the system 100 may comprise a plurality of cloud-based components, systems, or devices; while in other instances, the system 100 may comprise non-cloud-based systems or a combination thereof.

In one embodiment, a person who is required to conduct a self-testing or sampling at home or at any specific location may submit a request for a diagnostic kit via an online order system of the system 100 (Step 310). The order system can be provided in the form of a website, a software program, a web application, a mobile application, or the like. Prior to submission of the request, the user may have to register his/her personal information (Step 300) such as by creating a user account at the order system and the information will be stored securely at order system. The user information may include sensitive data such as medical records data in addition to simple or normal information such as delivery information, e.g., name and address information. The registration may be a one-off process and once the user account is registered, the user will be provided with a unique login for accessing her personal data which may include test request record, order, and test fulfilment status and/or diagnostic results, etc. Once the order request for a diagnostic kit is successfully submitted, an order identification (ID) will be created for the submitted order by the order system. The order identification will be recorded in the order system linking to the user's account. The order ID is preferably a unique ID.

An entity controlling the order system, after receiving the order request, may then release the requested diagnostic kit from the warehouse and dispatch the diagnostic kit 10 via a fulfillment service provider (Step 320). The entity controlling the order system may package the diagnostic kit 10 before releasing the requested diagnostic kit to the fulfillment service provider. Alternatively, the fulfillment service provider may be required by the entity controlling the order system to package the requested diagnostic kit prior to it being sent to the user. In any event, the requested diagnostic kit is packaged prior to being sent to a user. The entity controlling the order system and the fulfillment service provider may comprise one entity serving both functions, or the fulfillment service provider may be a separate entity under contract with the entity controlling the order system. With the packaged diagnostic kit 10 being provided with the identifier 40 at the packaging 20, the fulfilment service operator will arrange to scan, such as via a scanner 110, the identifier 40 (Step 330) provided by the packaging 20 of the diagnostic kit 10 to obtain the shipment details, e.g., the name and address of the user requesting the diagnostic kit. Particularly, the identifier 40 is configured such that it links to stored personal data of the user whom the diagnostic kit is to be delivered. The user's personal data is stored securely in the order system or an associated system. The user's personal data is stored such that access to it must be authorized, i.e., the data is not freely available to other entities other than the entity controlling the order system. Scanning of the identifier 40 provides the fulfilment service operator access only to delivery details of the user, such as name and address, to allow shipping of the packaged diagnostic kit to the user, but not any other stored personal data of the user such as, for example, medical records data. In one embodiment, the scanning of the identifier 40 will provide the fulfilment service operator only a one-time access to the delivery details of the person, such that the delivery details are displayed for a few seconds at the connected computing device to enable a delivery label or the like to be prepared by, for example, printing. Optionally, after scanning the identifier 40, the delivery details will be displayed once for printing via a printer or a printing module 140 of a computing device. The delivery details may then be printed out in the form of a postal label which is to be applied to the packaging 20 of the diagnostic kit 10 for shipping (Step 340). In one embodiment, the scanner 110 and the printer 140 can be provided in the form of one or more portable devices adapted to communicate wirelessly with the system 100. Alternatively, the scanner 110 and the label printer 140 can be integrated components of a fulfilment station operated by one or more operators.

The identifier 40 is configured such that it links to the order identification of the order request for the diagnostic kit. As described above, the order identification, when issued, is linked with the stored personal data of the user registered under the user account, which is managed and controlled securely by the order system. A user who is required to conduct multiple or repeated testing such as one who is under quarantine and is required to self-test regularly over a period of time is therefore not required to register his or her personal details for every test kit, as the order fulfillment service providers such as the courier service or test service will be able to access the user's information at a preset, limited level of disclosure sufficient to allow each party to fulfill the required order or testing procedure by just scanning the identifier 40 at the packaging 20 of the diagnostic kit 10 or other identifier at the test kit 30, which will be described further below. Furthermore, the user is not required to register his or her personal details after received the test kit 30.

The order identification is further linked to a kit identification (ID) 50 provided at the test unit 30 of the diagnostic kit 10, i.e., the testing device and/or device components for use by the user to conduct the self-testing or sampling. In one embodiment, the kit identification 50 provided at the test unit 30 of the diagnostic kit 10 is a unique identifier which may comprise a two-dimensional code, such as one or more of a quick response (QR) code, a bar code and/or any equivalent. Alternatively, the kit identification can be provided on one or more printed labels to be attached to the test unit 30 or components of the test unit 30, such as a sampling tube, a casing for the sampling tube, and/or a packaging for the casing, etc. after the self-sampling by the user.

As the personal data of the user is securely managed and controlled by only the order system, it is a requirement that no entity other than the entity controlling access to the order system is able to access the stored person data of the user linked to the order identification using the kit identification 50, and that any entity wishing to access the stored personal data of the user using the kit identification 50 must first obtain authorization from the entity controlling access to the order system. The present invention therefore ensures that no sensitive personal information will be disclosed to or be accessible by other entities or parties including the fulfilment service provider and/or the testing laboratories to protect privacy of at least the user's sensitive personal data.

In one embodiment, the user may obtain information about the ordered diagnostic kit 10 such as the diagnostic kit lot number, the stock keeping unit (SKU) code, the diagnostic kit expiry date and possibly also the diagnostic kit identification (ID) 50. In other embodiments, additional information may be obtained such as product name, the manufacturer of the diagnostic kit, the product specification, the application instruction, the product number, and the date of manufacture and of the diagnostic kit, etc. by scanning the identifier 40 at the packaging 20 and/or the kit identification at the test unit 30 using his or her own computer device equipped with a QR code reader such as a smart phone.

After the diagnostic kit 10 has been shipped to the user and the user has completed the sampling or testing, the test unit 30 with the collected specimen will be delivered to the designated testing service provider such as a laboratory or testing center. The designated testing service provider such as a laboratory or testing center may comprise part of the entity controlling the order system or may comprise a third-party entity under contract to the entity controlling the order system. Upon receipt of the used test unit 30, the laboratory technician may scan the kit identification 50 at the test unit 30 to obtain the unique kit identification (ID) 50. In some embodiments, the unique kit identification (ID) 50 may be used, where permission is granted, to access another system to determine which tests are to be run on the user's sample and some demographics of the user relevant to the tests to be run such as, for example, sex and age as these may be required to interpret the results obtained from the tests run. Such look-up may be done at the designated testing service provider's system but only if its system has previously been provided with such data prior to receiving the sampled test unit 30 from the user. Furthermore, in some embodiments, the unique kit identification (ID) 50 may encode the user's name so that an identity verification of the user can be performed by at the designated testing service provider but again only where the designated testing service provider's system has previously been provided with such data prior to receiving the sampled test unit 30 from the user. Scanning of the kit identification 50 may further allow the technician to record diagnostic data such as the type of test conducted and the test results (Step 350), with the recorded diagnostic data being linked further to the order identification and the user's account. Again, no entity other than the entity controlling access to the order system can access the recorded diagnostic data using the kit identification once such data is reported by the designated testing service provider, and any entity wishing to access the recorded diagnostic data using the kit identification must first obtain authorization from the entity controlling access to the order system. As the packaging for the requested test kit 30 sent to the user will have been disposed of by the user, no entity (other than the entity controlling the order system) handling the used test unit 30 after it has been dispatched by the user will even have access to the user's name and address as it is not required for the user to use this information when dispatching the used test unit 30 to the designated testing service provider.

In one embodiment, the kit identification 50 at the test unit 30 can be identical to the identifier 40 at the packaging 20 of the diagnostic kit 10, and that the level of permission for one to access the stored or linked information by scanning the same identifier 40/50 will be based on the access granted by the entity controlling access to the order system based on the individual's role in the order fulfilment process. Again, one may need to seek authorization from the entity controlling access to the order system to retrieve certain information such as to be provided with a specific passcode to login to the order system for accessing (limited) information and/or uploading information to the order system.

The present invention may further relate to a non-transitory computer readable medium storing machine-readable instructions which, when executed on a processor 120, implement the steps of method as described above.

While the invention has been illustrated and described in detail in the drawings and foregoing description, the same is to be considered as illustrative and not restrictive in character, it being understood that only exemplary embodiments have been shown and described and do not limit the scope of the invention in any manner. It can be appreciated that any of the features described herein may be used with any embodiment. The illustrative embodiments are not exclusive of each other or of other embodiments not recited herein. Accordingly, the invention also provides embodiments that comprise combinations of one or more of the illustrative embodiments described above. Modifications and variations of the invention as herein set forth can be made without departing from the spirit and scope thereof, and, therefore, only such limitations should be imposed as are indicated by the appended claims.

In the claims which follow and in the preceding description of the invention, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments of the invention.

It is to be understood that, if any prior art publication is referred to herein, such reference does not constitute an admission that the publication forms a part of the common general knowledge in the art.

1. A computer implemented method for managing order fulfilment of a diagnostic kit, comprising:
scanning, by a fulfilment entity, an identifier provided on a packaging of a diagnostic kit, the identifier being linked to stored personal data of a person to whom the diagnostic kit is to be delivered;
wherein the scanning of the identifier provides the fulfilment entity access only to delivery details of the stored personal data of the person so that the diagnostic kit can be delivered to the person.

2. The computer implemented method according to claim 1, wherein the scanning of the identifier provides the fulfilment entity only a one-time access to the delivery details of the person.

3. The computer implemented method according to claim 1, wherein the identifier is linked to an order identification of an order for the diagnostic kit, and the order identification is further linked to the stored personal data of the person to whom the diagnostic kit is to be delivered.

4. The computer implementing method according to claim 3, further comprising creating the order identification of the order for the diagnostic kit by an order system receiving the order prior to the scanning step.

5. The computer implementing method according to claim 4, wherein the diagnostic kit is provided with a kit identification linked to the order identification.

6. The computer implemented method according to claim 5, wherein no entity other than an entity controlling access to an order system receiving the order for the diagnostic kit can access the stored person data of the person linked to the order identification using the kit identification.

7. The computer implemented method according to claim 6, wherein an entity wishing to access the stored personal data of the person linked to the order identification using the kit identification must first obtain authorization from the entity controlling access to the order system which received the order for the diagnostic kit.

8. The computer implemented method according to claim 1, wherein the identifier provided on the packaging of a diagnostic kit comprises a two-dimensional code.

9. The computer implemented method according to claim 8, wherein the identifier provided on the packaging of a diagnostic kit comprises one or more of a quick response (QR) code and/or a bar code.

10. The computer implemented method according to claim 5, wherein the kit identification provided at the diagnostic kit comprises a two-dimensional code.

11. The computer implemented method according to claim 10, wherein the kit identification provided at the diagnostic kit comprises one or more of a quick response (QR) code and/or a bar code.

12. The computer implemented method according to claim 5, wherein the kit identification may further link to information of the diagnostic kit comprising one or more of: diagnostic kit lot number, stock keeping unit (SKU) code, diagnostic kit expiry date, diagnostic kit identification (ID), product name, manufacturer of diagnostic kit, product specification, application instruction, testing instruction and protocol, product number, date of manufacture of the diagnostic kit.

13. The computer implemented method according to claim 5, further comprising scanning the kit identification by an entity to record diagnostic data, wherein the recorded diagnostic data is linked to the order identification.

14. The computer implemented method according to claim 13, wherein no entity other than an entity controlling access to the order system receiving the order for the diagnostic kit can access the recorded diagnostic data using the kit identification.

15. The computer implemented method according to claim 14, wherein an entity wishing to access the recorded diagnostic data using the kit identification must first obtain authorization from the entity controlling access to the order system which received the order for the diagnostic kit.

16. A system for managing order fulfilment of a diagnostic kit, comprising:
a scanning module for scanning an identifier provided on a packaging of a diagnostic kit, a memory for storing data, and a processor for executing computer readable instructions, wherein the processor is configured by the computer readable instructions when being executed to implement the method of:
scanning, by a fulfilment entity, an identifier provided on a packaging of a diagnostic kit, the identifier being linked to stored personal data of a person to whom the diagnostic kit is to be delivered;
wherein the scanning of the identifier provides the fulfilment entity access only to delivery details of the stored personal data of the person so that the diagnostic kit can be delivered to the person.

17. A diagnostic kit, comprising:
a packaging where the diagnostic kit is packed, the packaging being provided with an identifier linked to stored personal data of a person to whom the diagnostic kit is to be delivered;
wherein scanning of the identifier provides a fulfilment entity access only to delivery details of the stored personal data of the person so that the diagnostic kit can be delivered to the person.
